(19) United States
(12) Patent Application Publication
(10) Pub. No.: US 2023/0140399 A1
(43) Pub. Date: May 4, 2023

(54) ACTIVE CHAIR ACTIVITY TRACKING SYSTEM

(71) Applicant: Kinetic Furniture of Vermont, Inc., Colchester, VT (US)

(72) Inventor: Turner Osler, Colchester, VT (US)

(21) Appl. No.: 17/820,739

(22) Filed: Aug. 18, 2022

Related U.S. Application Data
(60) Provisional application No. 63/263,346, filed on Nov. 1, 2021.

(51) Int. Cl. A61B 5/11; A61B 5/00

(52) U.S. Cl. CPC A61B 5/1116; A61B 5/1118; A61B 5/6891; A61B 5/742; A61B 5/4866; A61B 2562/0219

(57) ABSTRACT

A user's motion while seated on an active chair is monitored and the user's metabolic expenditures stemming from the motion are determined. Information associated with or derived from the measured motion and/or determined metabolic expenditures maybe be displayed. Motion sensors and gyroscopes are configured to detect the motion of the active chair associated with active sitting while the user is sitting. Data from these sensors is used to determine sitting activity of the user. In addition, representations of the movement of the active chair can be displayed on a screen in real time, allowing for the gamification of sitting as a way to encouraging users to move more, or to move in prespecified ways so as to learn new movement patterns and new skills.

### FIELD OF THE INVENTION

The present invention relates to seating devices. In particular, the present invention provides systems and methods for tracking activity of a user of an ergonomic seat.

### BACKGROUND

Human bodies are built to move and generally require constant activity to remain supple and healthy. Unfortunately, modern life involves a good deal of sitting. In fact, many professions require many hours of simply sitting, which puts an unnatural demand on the human body—so unnatural that children instinctively rebel against it.

Sitting, and especially sitting still, aligns human bodies oddly, and denies joints the constant small adjustments that help to circulate the joint fluid which helps nourish the delicate cartilage lining of the joints. Additionally, sitting still denies core muscles the exercise involved in aligning and realigning our spines, exercise vital to keeping our core musculature strong and responsive. Moreover, extended and repetitive sitting has been linked to other health maladies. Indeed, the mismatch between our 21st-century-built environment and our hunter-gatherer-optimized bodies has led to a variety of serious health problems: obesity, diabetes, heart disease, and even cancer, a litany of acquired diseases that can culminate in early death. These effects are not subtle—with a 50% increase in cancer risk among those who sat the most. Perhaps more importantly, simply reducing sitting could increase our lifespans by as much as two years.

One potential solution to these health issues may be the use of active chairs, which are designed to cause users to sit actively and thus may allow for the recapture of the healthy blood chemistry of our hunter-gatherer forebears. These chairs allow for movement while sitting, and so allow us to sit all day as our modern lives require without suffering the harm brought on by sitting inertly. An example of such an active chair is described in commonly owned U.S. Pat. No. 10,010,758.

A way to monitor or quantify the motion induced by sitting in such active chairs is to measure the motion.

### SUMMARY OF THE DISCLOSURE

A system for tracking sitting activity of a user includes an active chair with a seat, a rocking mechanism, and a base. A motion sensor is configured to detect motion of the seat about the rocking mechanism and a gyroscope is configured to determine a change in orientation of the seat. A processor is operatively connected with a memory, the motion sensor, and the gyroscope, and the memory stores computer-executable instructions for causing the processor to receive information from the motion sensor and the gyroscope, analyze, during a user siting session, the information from the motion sensor and gyroscope to determine whether the seat has undergone motion indicative of active sitter motion, and determine, based on a total amount of motion indicative of active sitting motion during the user sitting session, a number of calories used by the user during the user sitting session.

A method for tracking active sitting includes obtaining motion information from a motion sensor configured to detect motion associated with active sitting motions of an active chair when a user is seated on the active chair, obtaining orientation information from a gyroscope configured to detect a change in orientation of a seat of the active chair when the user is seated on the active chair, identifying, based on the motion information and the orientation information, by a processor, whether the seat has undergone active sitter motion, determining a total amount of active sitting motion during an active sitting session, and determining a number of calories used based on the total amount of active sitting motion during the user sitting session.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of an active chair with an attached motion sensor in accordance with an embodiment of the present invention;

FIG. 2 is a flow diagram of a method of measuring physical activity of a sitting user in accordance with an embodiment of the present invention;

FIG. 3 is a flow diagram of a method of determining metabolic expenditure of a sitting user in accordance with an embodiment of the present invention;

FIG. 4 is an illustration of a display screen with active sitting data in accordance with an embodiment of the present invention;

FIG. 5 is a perspective view of an active chair with a user seated on the active chair wherein a motion sensor is held by and associated with the user in accordance with another embodiment of the present invention;

FIG. 6 is a flow diagram of a method of measuring physical activity of a sitting user having a motion sensor in accordance with an embodiment of the present invention; and

FIG. 7 is a flow diagram of another method of measuring physical activity of a sitting user having a motion sensor in accordance with an embodiment of the present invention;

FIG. 8 a flow diagram for measuring physical activity of a sitting user having a motion sensor in accordance with an embodiment of the present invention; and

FIGS. 9A-9C are illustrations depicting a series of screenshots from a display screen displaying a target and a representation of seat position being updated in real time in accordance with another aspect of the invention.

### DESCRIPTION OF THE DISCLOSURE

The present invention provides for measuring a sitting user's motion associated with active sitting movements while seated on an active chair, determining the user's metabolic expenditures stemming from the sitting user's motion, and displaying information associated with or derived from the measured motion and/or determined metabolic expenditures. A sensor, such as an accelerometer and/or gyroscope, may be attached to an active sitting chair or attached to the sitter, such as by a strap or in a pocket of the sitter. The sensor is in communication with (or part of) an electronic device such as a smart phone. The motion detected by the sensor can be translated into data related to other forms of activity, such as calories burned. In addition, the detected motion can be used to create a real time position indicator on a display screen in which the position of the sensor is shown to move in sync with the sitter's motion on the active chair.

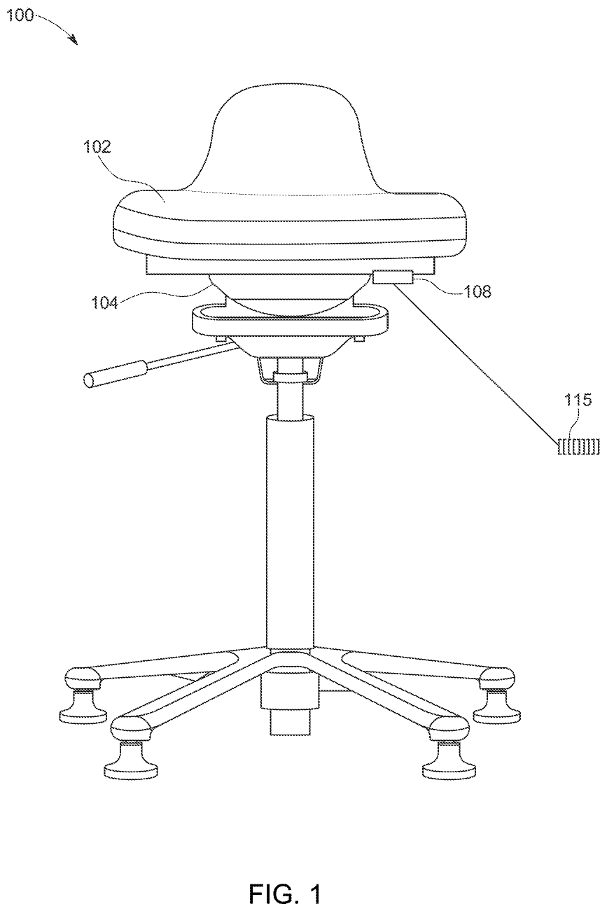

In an embodiment, an active sitting chair, such as active chair 100 shown in FIG. 1, includes a seat 102, a rocking or wobble mechanism 104 that allows for and encourages motion by a user sitting in chair 100, and one or more sensors 108 attached to chair 100. Sensors 108 may include an accelerometer and a gyroscope which may be attached at any appropriate location in order to detect acceleration and seat orientation along the X, Y, or Z axis (for example motion associated with the rocking and wobbling motion induced by wobble mechanism 104), such as beneath the seat as shown in FIG. 1. A 3-axis accelerometer could also be affixed to the person of the sitter, such as within a phone in a pocket of the sitter. Data from motion and orientation measured by sensors 108 may be processed by the accelerometer (e.g., if it is a smart sensor) or transmitted to a processor (preferably wirelessly) in an electronic device. Transmission may occur continuously, at intervals, or be collected after a period of sitting ends, and may be initiated by the sitter providing input that an active sitting session has begun.

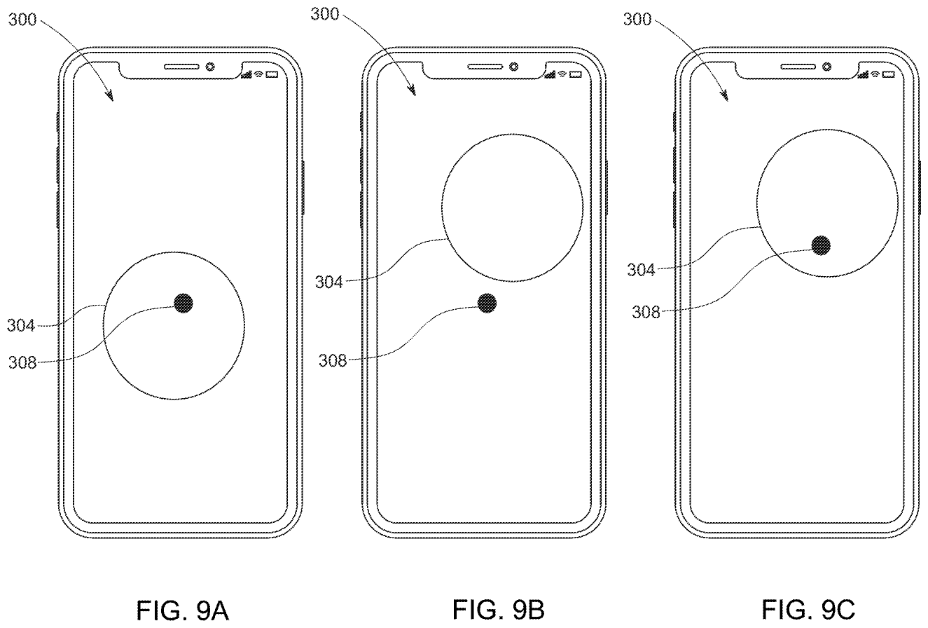

Motion data is used to determine a level of physical activity of the user sitting in chair 100. For example, data from sensors 108 is communicated to a processor 115 (shown schematically in FIG. 1) having a memory and may then be used to determine the frequency of motion, as well the amount of motion detected by the accelerometer along three axes, X, Y, and Z. This motion can be converted to calories burned by the sitter based on various parameters and associations, including the use of regression equations, for example, as well as the dimensions of chair 100 and the extent of motion enabled by wobble mechanism 104. In addition, tilt sensors gather data that can be used to display a representation of the movement of an active chair in real time on a display screen (as shown for example in FIGS. 9A-9C), allowing for the gamification of active sitting as a way to encourage users to move more, or to move in prespecified ways so as to learn new movement patterns.

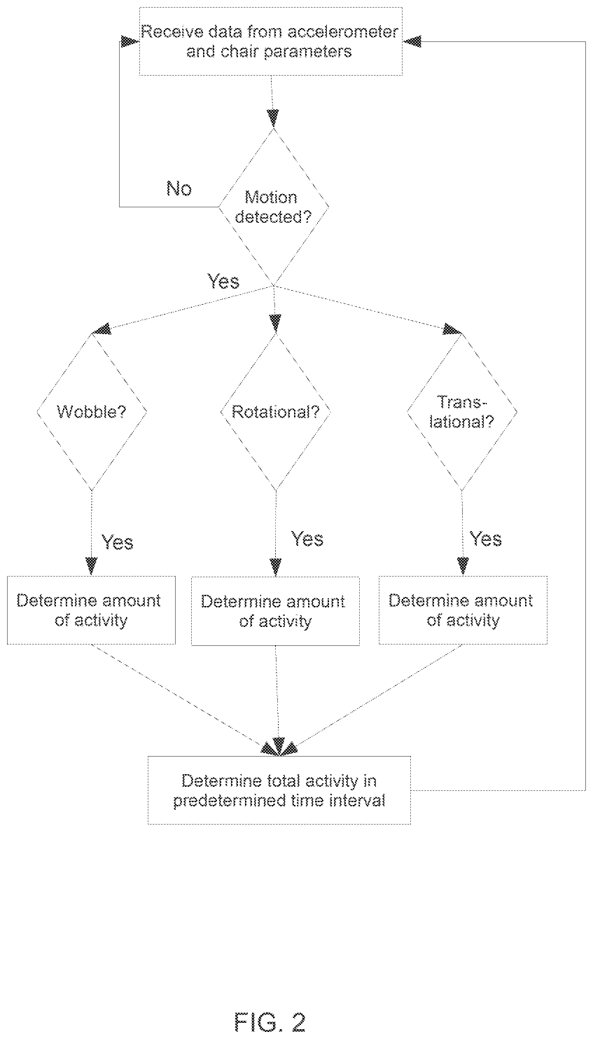

An exemplary process for determining activity level is outlined in FIG. 2. Data is received from the active chair accelerometer as well as the parameters for that active chair, such as seat size and wobble amount (e.g., tilt angle) allowed. It is then determined whether any motion was detected by the accelerometer. If no motion is detected, the process returns to the first step and waits to receive data from the accelerometer. If motion is detected, it may be converted to a measure of activity (e.g., counts). From those amounts of activity, the total amount of user sitting activity on the active chair that occurred during a predetermined time interval is determined. In addition, it may be determined whether the motion includes wobble, rotational, and/or translational motion, in which the amount of activity involved in creating such motion is determined based on the accelerometer data received along with the active chair parameters.

From the physical activity determined, metabolic expenditure of the user can be determined based further on information about the user, such as height, weight, age, etc. These parameters together with the measured chair activity levels can be used to estimate metabolic energy expenditures resulting from the active sitting. This may be based on calculations of energy required to perform such movements that result in the measured chair motion and/or measurements of physiologic parameters (such as respiration increases over baseline for similar individuals) that are correlated with the measured motion by the accelerometer.

From these increases in energy expenditure, i.e., caloric use increases compared to sitting in non-active chairs, may be determined for users as well as other outputs such as step equivalents or active chair sitting activity levels.

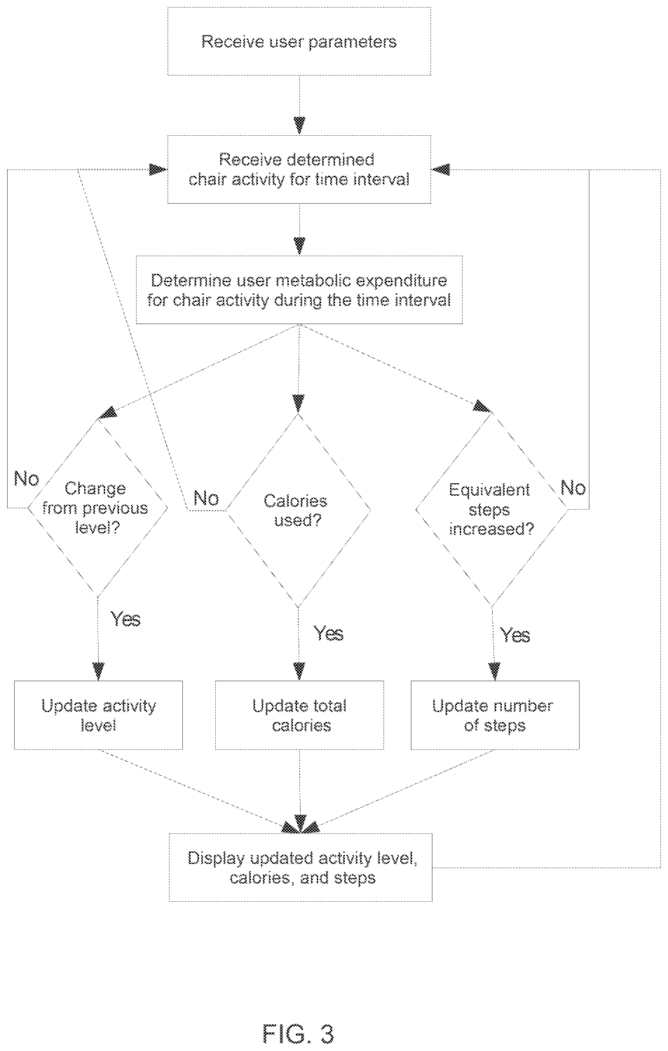

An exemplary process for determining user energy expenditure is outlined in FIG. 3. User parameters are received from input or other sources, and the total amount of user sitting activity on the active chair that occurred during a predetermined time interval is received. From the user parameters and the total amount of user sitting activity detected during an active sitting session, a user metabolic expenditure related to chair activity/motion is determined for the predetermined time interval. The user metabolic expenditure is then converted into desired outputs, such as sitting activity level, calories used from sitting activity, and equivalent steps. If none of these outputs change, in the next step the determined chair activity level is received for the next predetermined time interval. If any of these outputs changes during the most recent time interval, the outputs are updated and the displayed outputs are updated accordingly. At this point, the determined chair activity level is received for the next predetermined time interval.

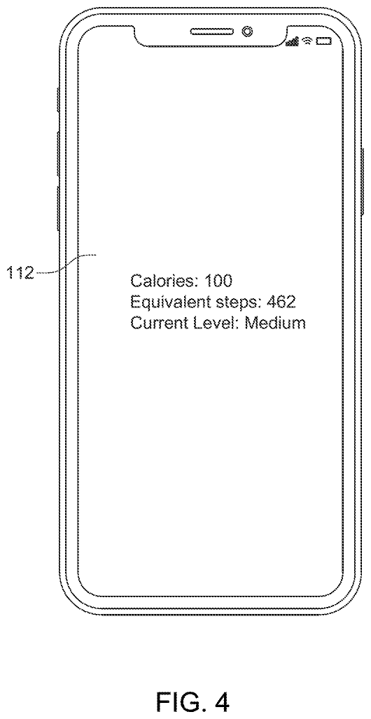

As shown in FIG. 4, a display 112 may be in communication with the processor to display, at the end of a sitting session or in real time, one or more of the active chair sitting activity outputs. These outputs may include total equivalent steps, total extra calories burned, current sitting activity level (e.g., low, medium, high). Display 112 may also be used to encourage user activity by displaying prompts or information linked to sensors 108, such as an icon that has its position on display 112 correlated with a current state of chair 100, such as a position and orientation of seat 102, based on data from sensors 108.

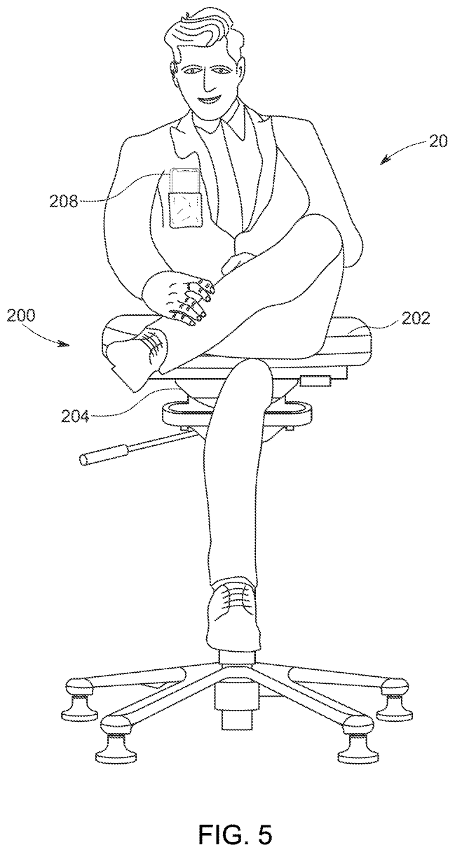

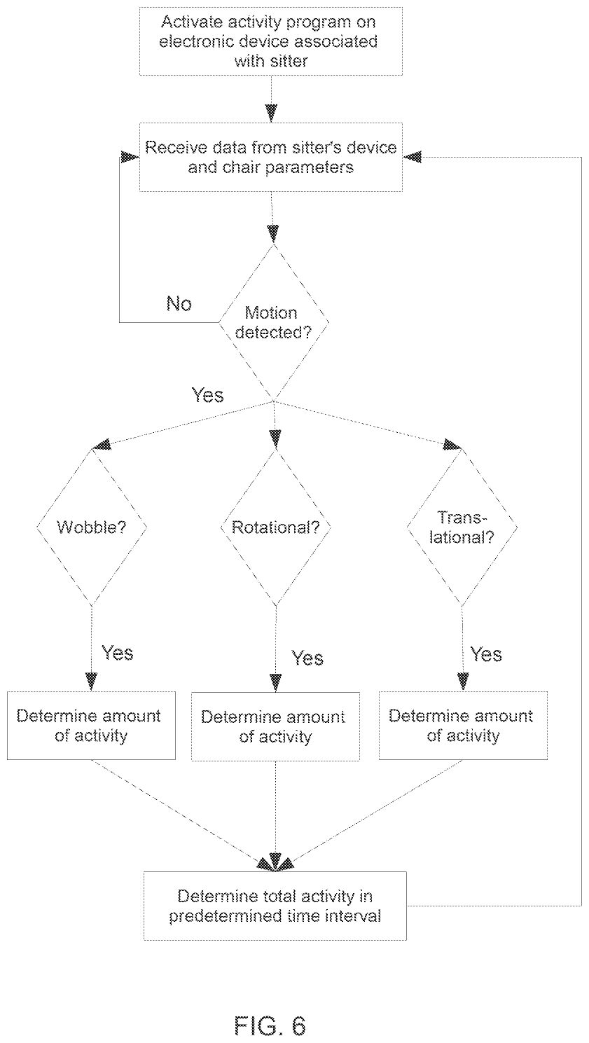

In another embodiment, the sensor/motion detector is located with the user/sitter, e.g., an accelerometer attached to the sitter or within an electronic device, such as a smart phone, held by a sitter, e.g., located in the sitter's pocket. FIG. 5 shows an active chair 200 having a wobble or rocker mechanism 204. A sitter 20 sits on a seat 202 of active chair 200 while carrying a device 208, such as a smart phone, in a pocket or otherwise attached to the sitter, that includes a motion detector such as an accelerometer and/or gyroscope. In a preferred embodiment, device 208 also includes an app or program that receives the detected motion data and determines the sitter's sitting activities from that data, as outlined in FIG. 6. Sitter and/or chair parameters may be entered into device 208 in order to facilitate more accurate determinations of activity and activity outputs (e.g., calories used for active sitting). The outputs may be transmitted via Bluetooth and displayed on the smart phone and/or on a device external to the smart phone such as a computer monitor and be updated at predetermined time intervals or based on certain changes in the outputs, e.g., changes in activity level or certain increases in calories used.

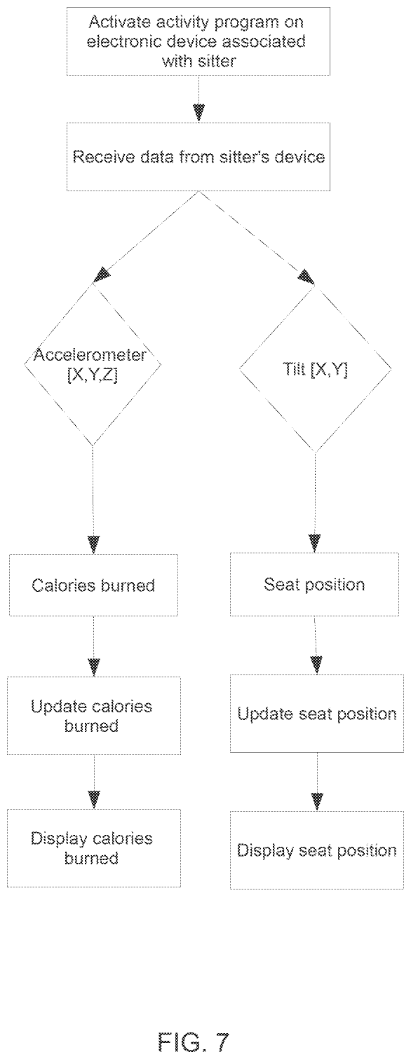

Another exemplary process for determining activity level and associated outputs is outlined in FIG. 7. When the program is activated by a user, data is received from the activity tracking sensors and motion detected by the accelerometer in any direction [X, Y, Z] is found as well as the tilt [X, Y]. From the motion data, calories burned are determined, updated, and displayed. From the tilt data, the current seat position is determined, updated and displayed. The seat position data may also be used to indicate a position on a monitor or within a window displaying an app in which an image or other image represents the current seat position with respect to the possible seat positions. This indication may be updated frequently, such as every 100 ms. In an embodiment, a maze or pattern could be shown that the sitter navigates with the seat position mark by controlling the position of the seat, or a target can be displayed that moves on the screen and the sitter causes the seat mark to follow the target by altering the position of the seat.

For any of the above embodiments, chair and user parameters are used to increase the accuracy of the determined outputs based on detected motions. For example, the type of chair will be associated with certain degrees and amounts of movement, which can inform the amount of energy associated with detected motions. Likewise, user parameters such as age and height allow these determinations to be further refined. In addition or in the alternative, detected motions may be associated with energy expenditure based on empirical analysis, e.g., associated with measured energy expenditure of similar users on similar chairs.

In addition to sensors attached to the active chair or the sitter and sensors incorporated into a smart phone or other device, the measuring, determining and/or displaying in accordance with the present invention may be integrated into a smartwatch or similar device.

The sitting activity outputs may be displayed on any of the above devices in real time, and cumulative data over a selected period of time (e.g., workday) may be provided, including via an activity versus time graph.

In an embodiment, an app allows a sitter to record and monitor a sitting session whenever they are sitting. Active siting movements are tracked through internal smart phone sensors or an external sensor, such as a BlueTooth sensor, and converts raw acceleration to caloric burn. The app may also display the tilt of the sensor, representing the user's tilt on the chair, and includes a game that requires the user to tilt the chair in specific directions to follow a target.

The app includes stores, which may include an external sensor store, a motion store, a data store and a user store. The external sensor store accesses the external sensor data through an external sensor module and the motion store accesses the phone's acceleration and gyroscope data from internal sensors using a motion module. Both the motion store and the external sensor store contain a timer, and an increment function that is called at each timer increment when the user has started a sitting session. The increment may be any suitable amount, such as every 0.02 seconds, which provides a frequency of accelerometer data of 50 Hz. In operation, each data point is appended to an array, and at every minute, this array is passed through several functions to process and save the data, and the array is cleared for the next minute of data. Various functions convert the raw data to counts, which may be a unitless measure of acceleration.

The counts may be converted to calories by any suitable method including based on the potential energy method. The determined total calories burned for that minute are saved, may be displayed, and are accumulated during the session.

Alternatively, the total calories used for active sitting may be determined by relating active sitting motion counts per unit time to a basal metabolic rate (BMR) associated with the user. For this determination, for example, every 1 count per minute detected of sitting activity, the user's BMR is considered to increase by about 10%. Thus, the total calories burned may be found by determining the counts per minute, relating that value to an increase in BMR, and summing the total additional calories used for the time periods in a sitting session. In another alternative, calories for a siting session may be determined based on the total distance traveled by the center of mass (of the sitter/chair) or based on the changes in the square of velocity of the sitter/chair about the tilt motion of the active chair. For example, the distance traveled may be determined based on the change of position of the sensor from one point in time to another. The velocity along that direction would be that distance divided by the time period between the points in time. Velocities may be determined in this manner for one or more directions (e.g., x axis and y axis) and the differences in determined velocities in a given direction or axis can be determined and squared and multiplied by one half the mass of the chair/sitter to yield an energy. The energies can be summed over all the measured velocity changes during a given sitting session to determine a total energy expenditure attributable to sitting motions during that sitting session.

The user's data is stored in the data store. This data may be used to create and maintain various variables representing the charts data and other user data.

Each of these stores are accessible in the app's views so that their data can be accessed and presented to the user. They may be initialized once at the launch of the app so that all views have access the same store, and therefore the same information.

In another aspect of the invention, all active sitting activities are monitored and accumulated based on the detection and distinctions of active sitting in comparison to other types of motion, such as walking or running. In this way, the user does not need to provide input to start a session when first sitting or stop a session when getting up from the active chair. For example, the program may distinguish (such as via machine learning) the type (e.g., based on direction, frequency, magnitude) of motion associated with active sitting motion from the type of motion associated with other movements by a user, such as walking. Motion detected by an accelerometer on a user while walking will have a different pattern than motion detected by the accelerometer on the user while engaged in active sitting.

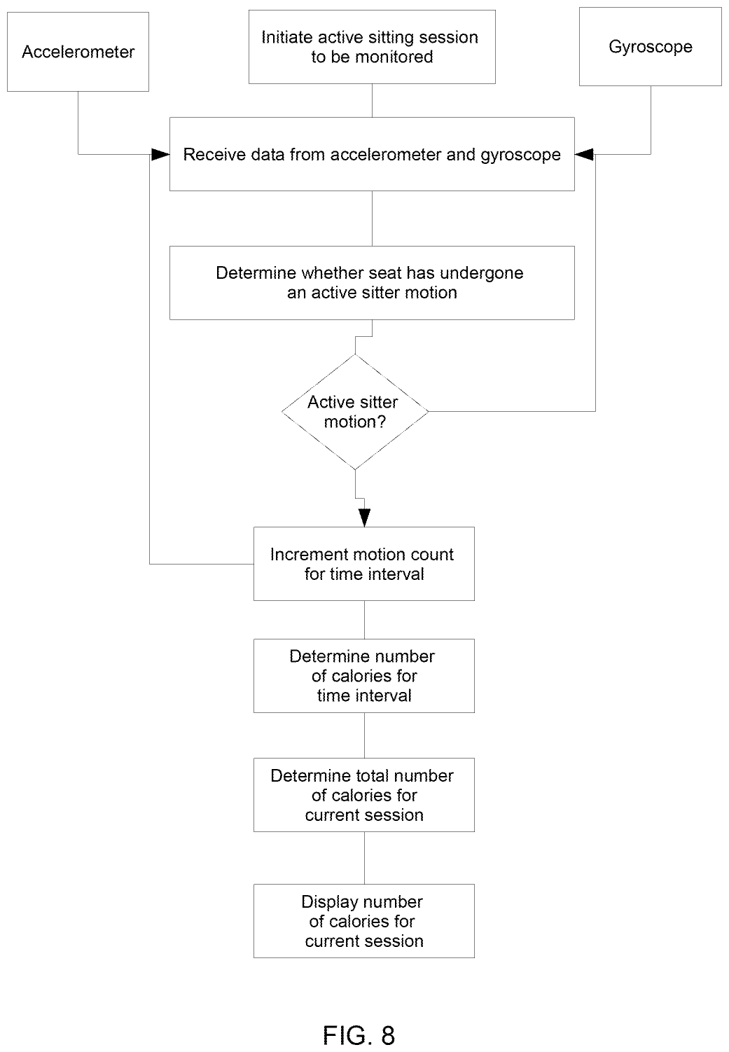

Another exemplary process for determining activity level and associated outputs is outlined in FIG. 8 in which motion information is obtained from a motion sensor such as an accelerometer configured to detect motion of an active chair when a user is seated on the active chair and orientation information is obtained from a gyroscope configured to detect a change in orientation of a seat of the active chair when the user is seated on the active chair. Based on the motion information and the orientation information, it is determined whether the seat has undergone an active sitter motion associated with the type of motion induced by the active chair when the user is seated in the active chair. During a predetermined interval, a motion count, or other measure of sitting activity, total is incremented whenever the seat has undergone the active sitter motion. Then, based on the sitting motion total accumulated during a user sitting session, a number of calories used by the user during the user sitting session is determined (via one or more of the techniques described above) and may be displayed on a display screen as well as tracked as part of session, daily, monthly, etc. totals, which may include comparing to goals, past averages, and equivalents to other activities such as walking or swimming.

In FIGS. 9A-9C, a display screen 300 is shown that includes a representation 308 of a current position of a seat of an active chair based on data from sensors on the active chair or on the sitter in the active chair that is translated in real time to representation 308 of a relative position/orientation of the active chair seat that is sent to and displayed on display screen 300. Display screen 300 may also include a target 304 that is displayed along with representation 308 in order to challenge the sitter to control the position/orientation of the seat through active sitter movements that result in repositioning of representation 308. In FIG. 9B, target 304 is moved such that representation 308 is no longer within target 304. In FIG. 9C, the sitter has engaged in motion that caused representation 308 to move back within target 304.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for tracking sitting activity of a user comprising:
an active chair including a seat, a rocking mechanism, and a base;
a motion sensor configured to detect motion of the seat about the rocking mechanism;
a gyroscope configured to determine a change in orientation of the seat;
a memory; and
a processor,
wherein:
the processor is operatively connected with the memory, the motion sensor, and the gyroscope, and
the memory stores computer-executable instructions for causing the processor to:
receive information from the motion sensor and the gyroscope;
analyze, during a user siting session, the information from the motion sensor and the gyroscope to determine whether the seat has undergone motion indicative of active sitter motion, and
determine, based on a total amount of motion indicative of active sitting motion during the user sitting session, a number of calories used by the user during the user sitting session.

2. The system of claim 1, wherein the instructions cause the processor to increment, during a predetermined interval, a motion count stored in an array in the memory when the seat has undergone a threshold amount of motion indicative of active sitter motion.

3. The system of claim 2, wherein the number of calories used is determined based on the motion count.

4. The system of claim 1, wherein whether the seat has undergone motion indicative of active sitter motion is determine based on changes in velocity of the seat in a plurality of directions.

5. The system of claim 4, wherein the number of calories used is determined based on a total amount of energy associated with the changes in velocity of the seat.

6. The system of claim 1, wherein the user sitting session is initiated and terminated based on user inputs.

7. The system of claim 1, wherein the information includes data representing rocking motion, rotational motion, and translational motion.

8. The system of claim 1, wherein the memory stores user parameters and wherein the number of calories is further determined based on one or more of the user parameters.

9. The system of claim 1, wherein the motion sensor is attached to the active chair.

10. The system of claim 1, wherein the motion sensor and the gyroscope are attached to the user.

11. The system of claim 1, further including a display screen operably connected to the processor, wherein the display screen receives location data from the processor based on the information and displays a representation corresponding to a current state of the seat.

12. The system of claim 11, wherein the representation corresponding to the current state of the seat changes position on the display screen in two dimensions based on the information.

13. The system of claim 1, wherein the user sitting session is initiated and terminated based on instructions causing the processor to determine when the user sitting session begins and ends based on the information.

14. The system of claim 13, wherein when the user sitting session begins and ends is determined by detecting a start of sitting activity motion that is distinguishable from non-sitting activity motion based on the information.

15. A method for assessing sitting activity in an active chair comprising:
obtaining motion information from a motion sensor configured to detect motion associated with active sitting motions of the active chair when a user is seated on the active chair;
obtaining orientation information from a gyroscope configured to detect a change in orientation of a seat of the active chair when the user is seated on the active chair;
identifying, based on the motion information and the orientation information, whether the seat has undergone movement associated with active sitter motion;
determining a total amount of active sitting motion during an active sitting session; and
determining a number of calories used based on the total amount of active sitting motion during the user sitting session.

16. The method of claim 15, further including incrementing a motion count total when the seat has undergone active sitter motion during a predetermined interval.

17. The method of claim 16, wherein the number of calories is determined from the motion count total.

18. The method of claim 15, wherein whether the seat has undergone active sitting motion is determine based on changes in velocity of the active chair in a plurality of directions.

19. The system of claim 18, wherein the number of calories used is determined based on a total amount of energy associated with the changes in velocity.

20. The method of claim 15, further including initiating and terminating the user sitting session based on user inputs.

21. The method of claim 20, wherein the motion information includes data representing rocking motion, rotational motion, and translational motion.

22. The method of claim 20, further including storing user parameters, wherein the number of calories is further determined based on one or more of the user parameters.

23. The system of claim 20, further including attaching the motion sensor and the gyroscope to the user.

24. The system of claim 20, further including displaying on a display screen a representation corresponding to a current position and orientation of the seat of the active chair.

25. The system of claim 24, further including changing a location of the representation on the display based on the sensor information and the gyroscope information.
